(19) United States
(12) Patent Application Publication
(10) Pub. No.: US 2013/0184608 A1
(43) Pub. Date: Jul. 18, 2013

(54) METHODS AND SYSTEMS FOR DETECTING AND REMOVING METALLIC FOREIGN BODIES

(71) Applicant: Kevin Anthony Pezzi, Thompsonville, MI (US)

(72) Inventor: Kevin Anthony Pezzi, Thompsonville, MI (US)

(73) Assignee: EMPIRE TECHNOLOGY DEVELOPMENT LLC, Wilmington, DE (US)

(21) Appl. No.: 13/636,778

(22) PCT Filed: Jan. 12, 2012
(86) PCT No.: PCT/US12/21101

§ 371 Date: Sep. 24, 2012

(51) Int. Cl. A61B 5/06; A61N 7/00; A61B 17/52; A61M 1/00; A61M 37/00

(52) U.S. Cl. 600/550; 600/11

(57) ABSTRACT

Methods and systems for detecting and removing a metallic object from a subject are disclosed. A sheath may be inserted into a channel of the subject. The sheath may include a probe that is removably or movably positioned within the sheath. The probe may include a sensor positioned at or near a distal end of the probe. A location of the metallic object may be detected in the subject using the sensor. The metallic object may be caused to become affixed to one or more of the sheath and the probe. The affixed metallic object may be removed from the subject.

### BACKGROUND

Metallic foreign bodies can enter human or animal bodies from a variety of sources, such as bullets, shotgun pellets, air gun BBs or pellets, bomb fragments, shattered hammer or chisel fragments and/or objects ejected from various tools, such as high-speed rotary tools. Typically, surgeons attempt to remove foreign bodies by entering a wound channel created by the object as it entered the body. A foreign body extractor is often used to avoid extending the wound, which would create additional trauma to the subject.

Precisely locating the foreign body is difficult even for experienced surgeons. Typically, a surgeon obtains two x-rays of the affected area perpendicular to each other as a means of estimating the location of the object. However, x-rays are of limited value when attempting to locate small foreign bodies. Moreover, surgeons must still blindly probe inside the puncture wound, wasting time and creating additional trauma for the subject.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a”, “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a system for detecting and removing a metallic object from a subject may include a sheath having a distal end and configured to have the distal end inserted into a channel of the subject, a probe configured to be movably positioned within the sheath and including a sensor positioned at or near a distal end of the probe and configured to detect a metallic object, and a vacuum lumen positioned within the sheath and configured to provide suction at the distal end of the sheath.

In an embodiment, a system for detecting and removing a metallic object from a subject may include a sheath having a distal end and configured to have the distal end inserted into a channel of the subject, and a probe configured to be removably positioned within the sheath and including a sensor positioned at or near a distal end of the probe and configured to detect a metallic object.

In an embodiment, a system for detecting and removing a metallic object from a subject may include a sheath having a distal end and configured to have the distal end inserted into a channel of the subject and to removably receive a probe during a detection operation that includes a sensor positioned at or near a distal end of the probe and configured to detect a metallic object.

In an embodiment, a method of detecting and removing a metallic object from a subject may include inserting a sheath into a channel of the subject, where the sheath includes a probe movably positioned within the sheath and including a sensor positioned at or near a distal end of the probe, detecting, via the sensor, a location of the metallic object in the subject, causing the metallic object to become affixed to one or more of the sheath and the probe, and removing the sheath and the affixed metallic object from the subject.

In an embodiment, a method of detecting and removing a metallic object from a subject may include inserting a sheath into a channel of a subject, inserting a probe including a sensor positioned at or near a distal end of the probe into the sheath, detecting, via the sensor, a location of the metallic object in the subject, removing the probe front the sheath, causing the metallic object to become affixed to the sheath, and removing the sheath and the affixed metallic object from the subject.

In an embodiment, a kit may include a sheath configured to be inserted into a channel of a subject, a probe including a sensor positioned at or near a distal end of the probe and configured to be inserted within the sheath, and instructions for detecting and removing a metallic object from the subject using the probe and the sheath.

### BRIEF DESCRIPTION OF THE FIGURES

FIG. 1 depicts a circuit diagram for an illustrative sensor circuit according to an embodiment.

FIG. 2 depicts an illustrative metal detector and probe according to an embodiment.

FIG. 3A depicts an illustrative system for detecting a metallic object in a subject according to an embodiment.

FIGS. 3B-3F depict alternate illustrative systems for removing a metallic object from a subject according to embodiments.

FIGS. 4A and 4B depict illustrative detection and extraction operations using an illustrative system according to an embodiment.

FIG. 5 depicts an alternate illustrative extraction device for detecting and removing a metallic object in a subject according to an embodiment.

FIG. 6 depicts a flow diagram of an illustrative method of detecting and removing a metallic object according to an embodiment.

FIG. 7 depicts a flow diagram of an alternate illustrative method of detecting an removing a metallic object according to an embodiment.

### DETAILED DESCRIPTION

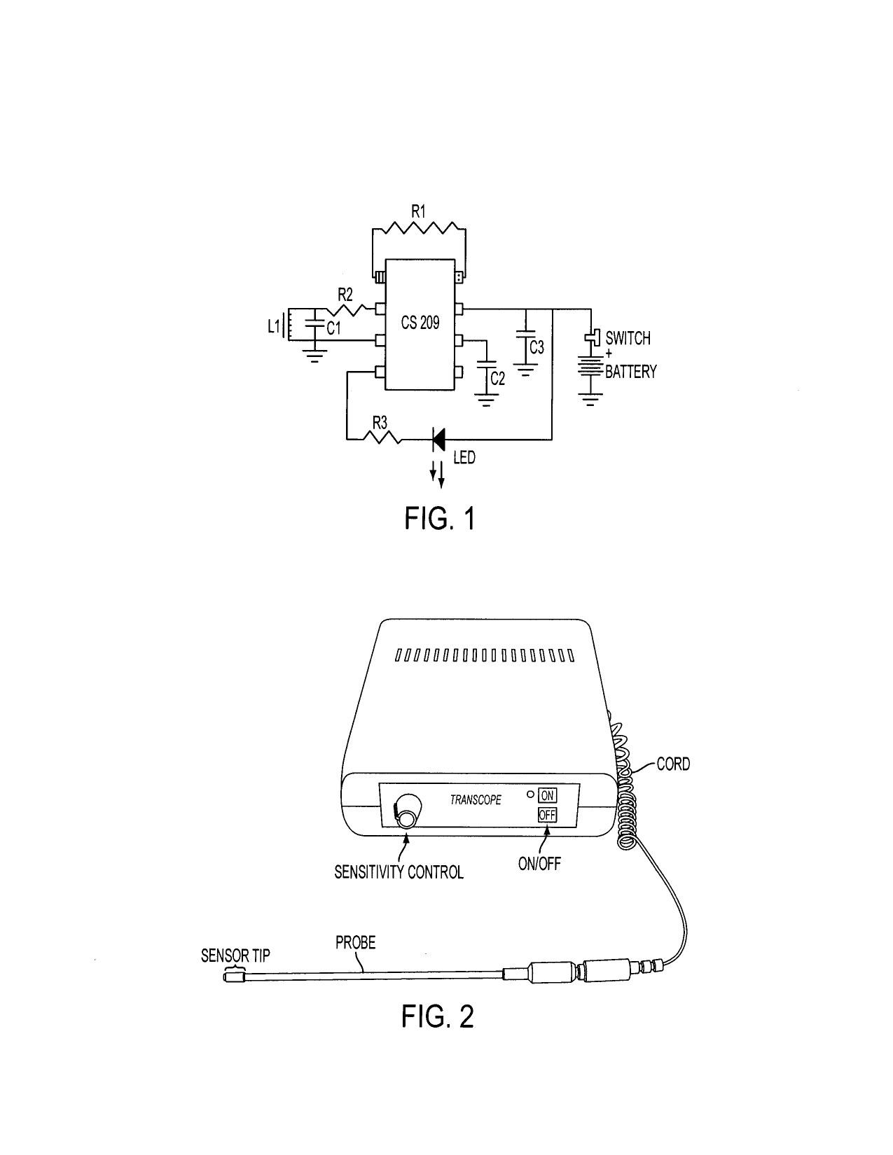

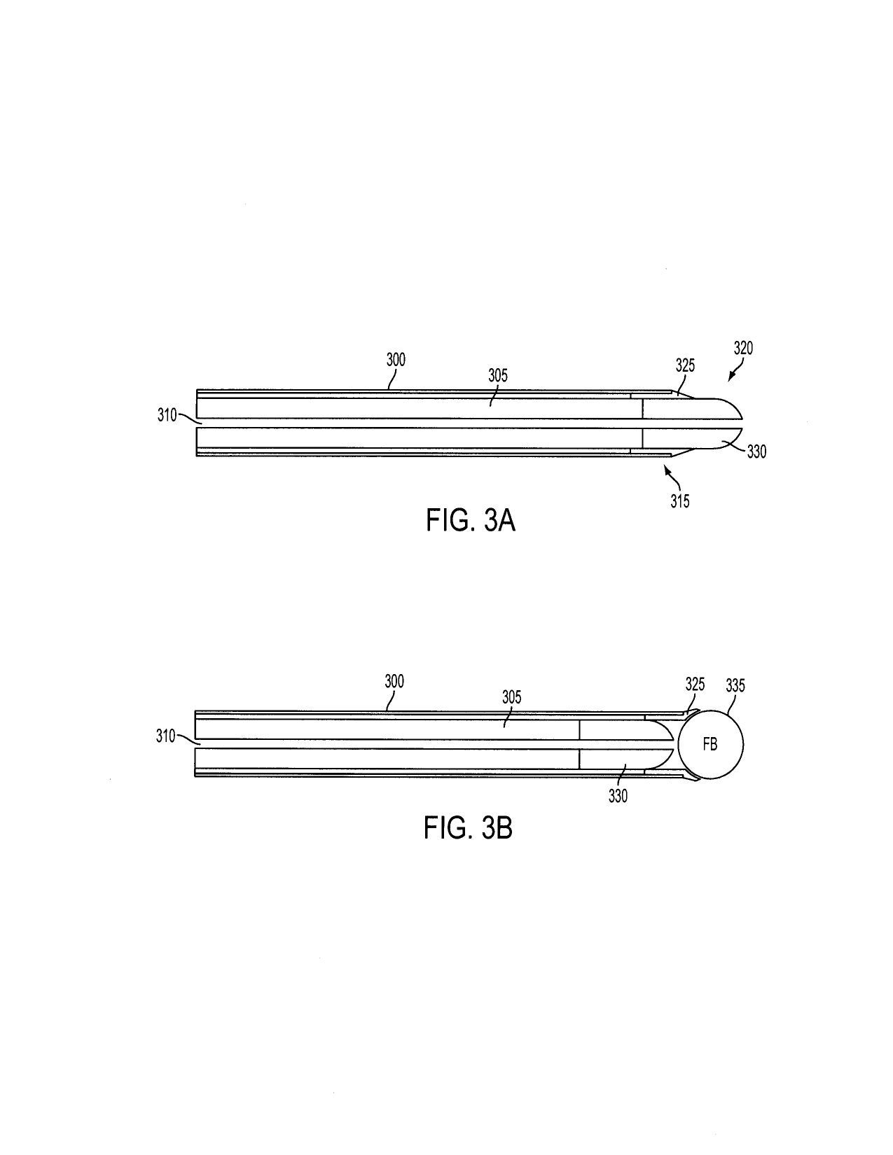

This disclosure identifies methods and systems for detecting and removing at least one metallic object from a subject. The systems disclosed herein employ a sensor used to detect the at least one metallic object in the subject. FIG. 1 depicts a circuit diagram for an illustrative sensor circuit according to an embodiment. As shown in FIG. 1, an illustrative sensor circuit may include an inductor L1, coupled to a proximity detector chip, such as the CS209 produced by Cherry Semiconductor of East Greenwich, R.I. Resistor R1 may be a variable resistor used to set the detection sensitivity for the sensor circuit. An alert system LED, such as a light emitting diode, a buzzer, and/or any other auditory or visual alert. The alert system LED may be activated hen a metallic object approaches within a particular distance of inductor L1. For example, if resistor R1 is adjusted to make the sensor circuit more sensitive, the sensor circuit ay detect metallic objects that are within about 15 mm of inductor L1. In contrast, if resistor R1 is adjusted to make the sensor circuit less sensitive, the sensor circuit may detect metallic objects that are within about 1 mm of inductor L1. Other higher or lower sensitivity distances may be obtained by adjusting the sensor circuit. Exemplary device values for the components of the sensor circuit depicted in FIG. 1 are as follows: R1—20 kΩ internal potentiometer+500Ω user sensitivity control, R2—200Ω, R3—680Ω, L1—200 μH, C1—0.002 μF polystyrene capacitor, C2—0.0022 μF, and C3—0.1 μF. In an embodiment, the battery ay provide a source voltage, such as 9 Volts, required by the proximity detector chip. FIG. 2 depicts an illustrative metal detector and probe that utilizes a sensor circuit according to an embodiment. The methods and systems disclosed herein may be used in conjunction with the above-described sensor circuit and/or additional and/or alternate sensor circuits within the scope of this disclosure. The sensitivity distances can be varied during use, where the distance can initially be relatively large, and can be reduced to a smaller distance as the probe more closely approaches the metallic object.

As used herein, the term “sensor” refers to at least the portion of a sensor circuit, such as inductor L1 in the sensor circuit described above, which is configured to be in close proximity to a metallic object during a detection operation. The sensor may refer to at least a portion of the sensor circuit that is inserted into a channel of a subject during a detection operation. Additional portions of the sensor circuit may be located at more distant locations from the metallic object to be detected during the detection operation.

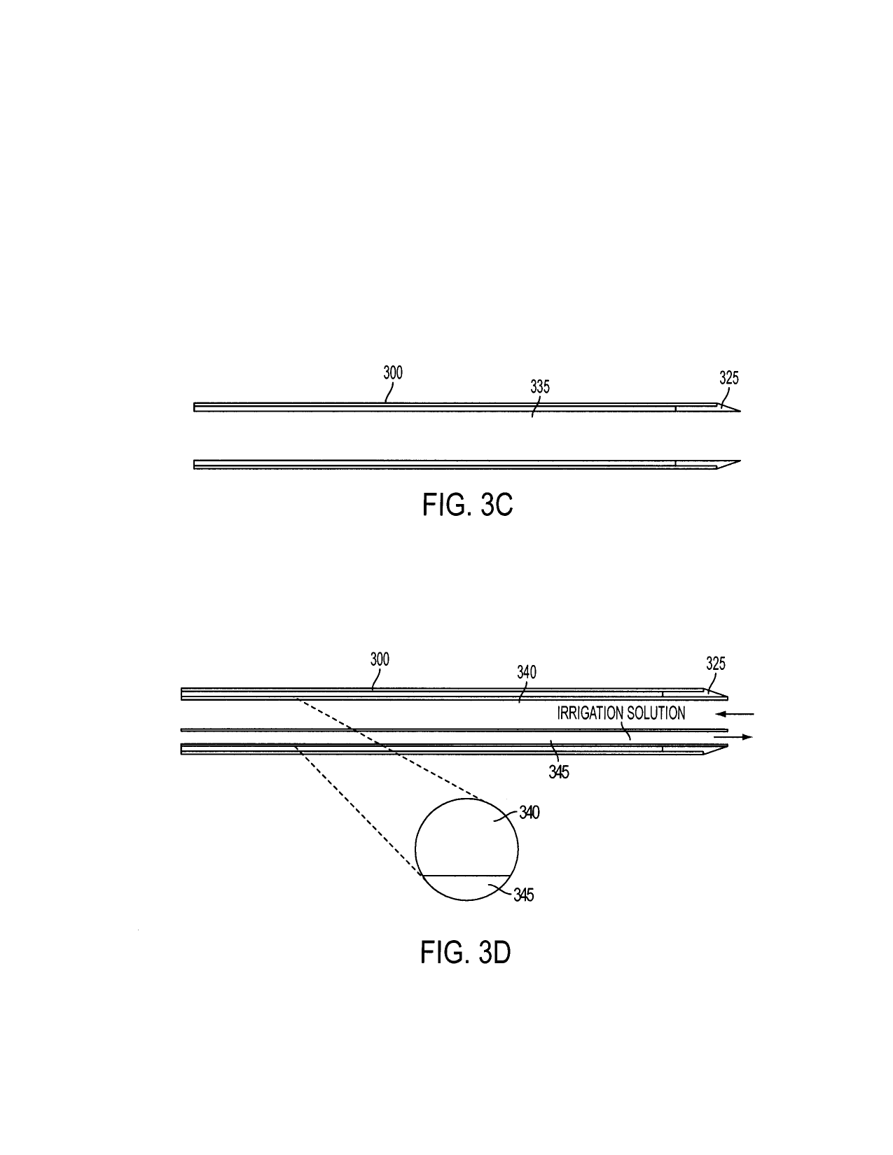

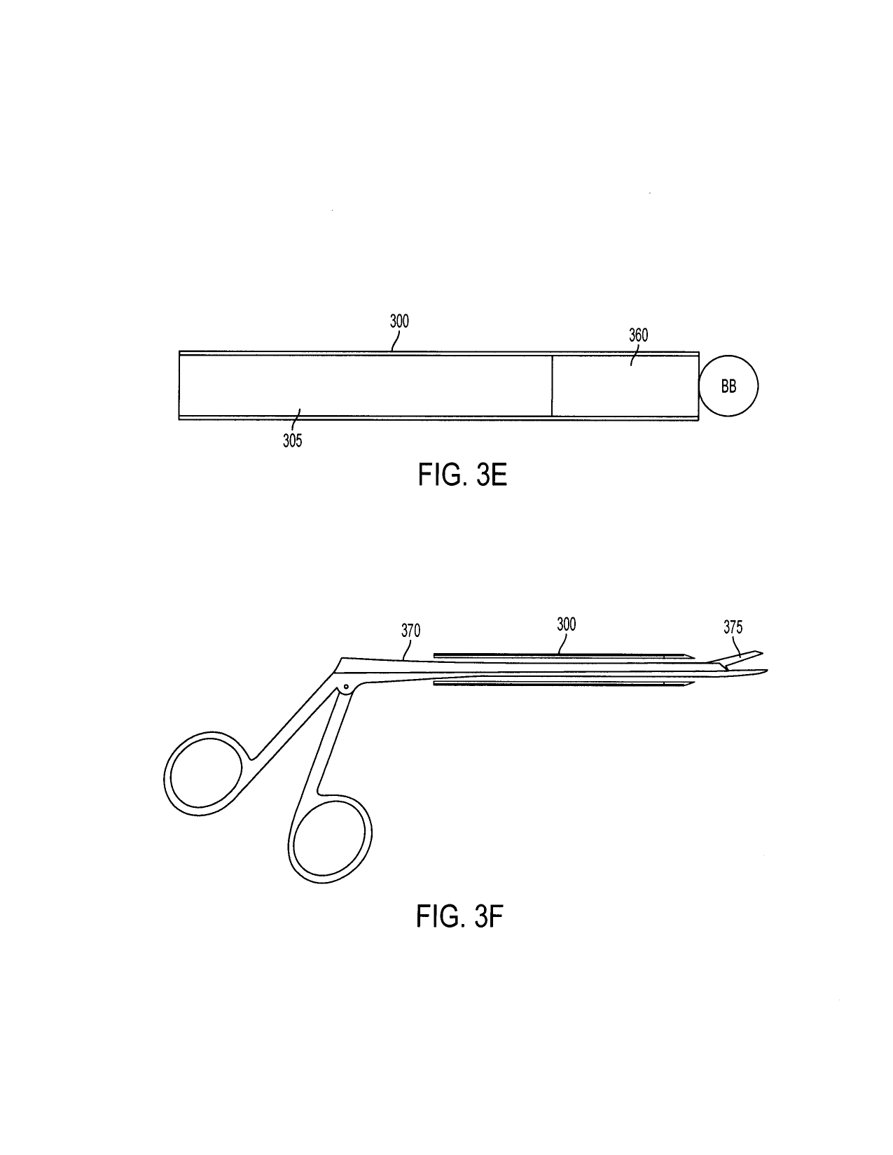

FIG. 3A depicts an illustrative system for detecting a metallic object in a subject according to an embodiment. As shown in FIG. 3A, a system for detecting and removing a metallic object from a subject may include a sheath 300, a probe 305, and a vacuum lumen 310. The sheath 300 may have a distal end 315 that is configured to be inserted into a channel of a subject, such as a wound channel.

The probe 305 may be configured to be movably positioned within the sheath 300. In an embodiment, a distal end 320 of the probe 305 maybe configured to be positioned outside of the distal end 315 of the sheath 300 during a detection operation, as is shown in FIG. 3A. The probe 305 may be moved into the distal position during the detection operation to enable the sensor to be more fully exposed to the metallic object. In an embodiment, the distal end 320 of the probe 305 may be configured to be recessed within the sheath 300 during a re oval operation, such as is shown in FIG. 3B. The probe 305 may be moved into a recessed position during the removal operation to enable suction to be caused through the vacuum lumen 310. In an embodiment, the sheath 300 or the probe 305 may include an elastomeric tip 325. In an embodiment, the elastomeric tip 325 may be positioned around at least a portion of the probe 305 during a detection operation, such as is shown in FIG. 3A. In an embodiment, the elastomeric tip 325 may be positioned distally fro the probe 305 during a removal operation, such as is shown in FIG. 3B. The elastomeric tip 325 may assist in sealing the suction applied via the vacuum lumen 310 to the metallic object, such as 335, thereby enabling the metallic object to be withdrawn fro the subject as the sheath 300 is removed.

The probe 305 may include a sensor 330 configured to detect a metallic object. The sensor 330 may be positioned at or near the distal end 320 of the probe 305. In an embodiment, the sensor 330 is configured to cause an alert to be produced when a metallic object is detected. The alert may include a visual alert, an auditory alert, or any other kind of discernible alert. In an embodiment, the alert may increase in intensity as the sensor 330 approaches the metallic object and may decrease in intensity as the sensor becomes more distant from the metallic object.

In an embodiment, a sensitivity of the sensor 330 may be configured to be controlled or varied by an operator. For example, the sensor 330 may be configured to detect a metallic object located about 0 mm to about 15 mm from the sensor. In an embodiment, the sensor 330 may be configured to detect a metallic object baying a mass of at least about 1 mg, at least about 5 mg, at least about 10 mg, at least about 20 mg or any other mass.

The vacuum lumen 310 may be positioned within the sheath 300 and may be configured to provide suction at the distal end 315 of the sheath. In an embodiment, the vacuum lumen 310 may be positioned through the probe 305.

FIG. 3C depicts an alternate system for removing a metallic object from a subject according to embodiments. As depicted in FIG. 3C, a probe may be configured to be completely removed from the sheath 300 during a removal operation. In an embodiment, the sheath 300 may be configured to provide a vacuum (i.e., suction) during the removal operation. In such an embodiment, the sheath 300 may operate as a vacuum lumen 335 during the removal operation. In an embodiment, the sheath 300 may include an elastomeric tip 325 configured to be positioned at a distal end of the sheath and to serve the function described above.

FIG. 3D depicts yet another system for removing a metallic object from a subject according to embodiments. As depicted in FIG. 3D, a vacuum lumen 340 may be inserted into the sheath 300 as part of a removal operation. The vacuum lumen 340 may be configured to provide a vacuum during a removal operation. In an embodiment, an irrigation channel 345 may also be provided within the sheath 300. The irrigation channel 345 may be configured to provide an irrigation solution during a removal operation. In an embodiment, the irrigation channel 345 may be inserted simultaneously with the vacuum lumen 340. In an embodiment, the vacuum lumen 340 and the irrigation channel 345 may each be part of a catheter inserted into the sheath. The irrigation solution provided through the irrigation channel 345 may be used to dislodge a metallic object that is embedded in tissue of the subject and to remove germs that could cause infection around the wound channel. Irrigation is particularly useful when the subject has small and/or numerous metallic or nonmetallic objects or contaminants located within it.

In an embodiment, a thermistor may be used. The thermistor may be configured to sense a temperature of the irrigation solution to prevent the irrigation solution from causing harm to the patient. In an embodiment, the thermistor may be located at a proximal end of the sheath 300, catheter or irrigation channel 345. In such an embodiment, the thermistor may be set to provide a warning if the temperature of the irrigation solution is more than slightly greater than a comfortable temperature for the subject because of heat loss during transmission of the irrigation solution through the irrigation channel. In an alternate embodiment, the thermistor may be located at a distal end of the sheath 300, catheter or irrigation channel 345. In such an embodiment, the thermistor may be set to provide a warning if the temperature of the irrigation solution is greater than a comfortable temperature for the subject. In an embodiment, a heating element may be used to heat the irrigation solution. The heating element may be positioned proximally to the thermistor and may receive feedback information from the thermistor to control the temperature of the irrigation solution.

In an embodiment, the probe 305 may include one or more depth markings. The depth markings may be configured to identify a depth to which the probe 305 is inserted into the sheath 300.

FIG. 3E depicts an illustrative probe with a magnet that may be used as a system for removing a metallic object from a subject according to an embodiment. As shown in FIG. 3E, the sheath 300 may be configured to receive a magnet 360, which is positioned at the distal end of the sheath during a removal operation. In an embodiment, the magnet may include, without limitation, a plated neodymium magnet. The magnet 360 may attach to metallic objects in the subject during a removal operation and may be removed through the sheath to draw the metallic objects from the subject. In an embodiment, the magnet 360 may be used in concert with a vacuum lumen 310, 335 or 340, irrigation channel 345 and/or other components described herein in order to enhance the ability to remove metallic objects. In an embodiment, the magnet 360 may be part of the probe 305 that is used to detect the metallic objects during the detection operation.

FIG. 3F depicts an illustrative extraction device inserted in an illustrative sheath according to an embodiment. As shown in FIG. 3F, the sheath 300 may be configured to receive an extraction device 370 during a removal operation. The extraction device 370 may be used to extract a metallic object by grasping the object and removing it from the subject either through the sheath 300 or as the sheath is removed from the subject. In an embodiment, the extraction device 370 may include a gripping tool 375 at a distal end of the device that is configured to grasp a metallic object. For example, the gripping tool 375 may be placed in an open configuration in which the gripping tool may be placed around a metallic object. The gripping tool 375 may then be placed in a closed configuration in which the gripping tool closes or partially closes to affix itself to the metallic object.

In an embodiment, the extraction device 370 may include a plurality of depth markings configured to identify a depth to which the extraction device is inserted into the sheath 300. These markings may be used in conjunction with markings on a probe 305 to more accurately determine the location of a metallic object in a subject. In an alternate embodiment, the probe 305 may include an extraction device 370 or the extraction device may be configured to be used as a probe.

In an embodiment, the sheath 300 may include a catheter. The catheter may include a vacuum lumen configured to provide a vacuum. In an embodiment, the catheter may further include an irrigation channel configured to provide an irrigation solution.

In an embodiment, the sheath 300 may include a magnet. In an embodiment, the sheath 300 may include an extraction device. In an embodiment, the sheath 300 may include an extraction device with a gripping tool at a distal end.

In an embodiment, the sheath 300, the probe 305 or any other component described above may include an ultrasound transducer. The ultrasound transducer may be configured to dislodge the metallic object using ultrasound waves.

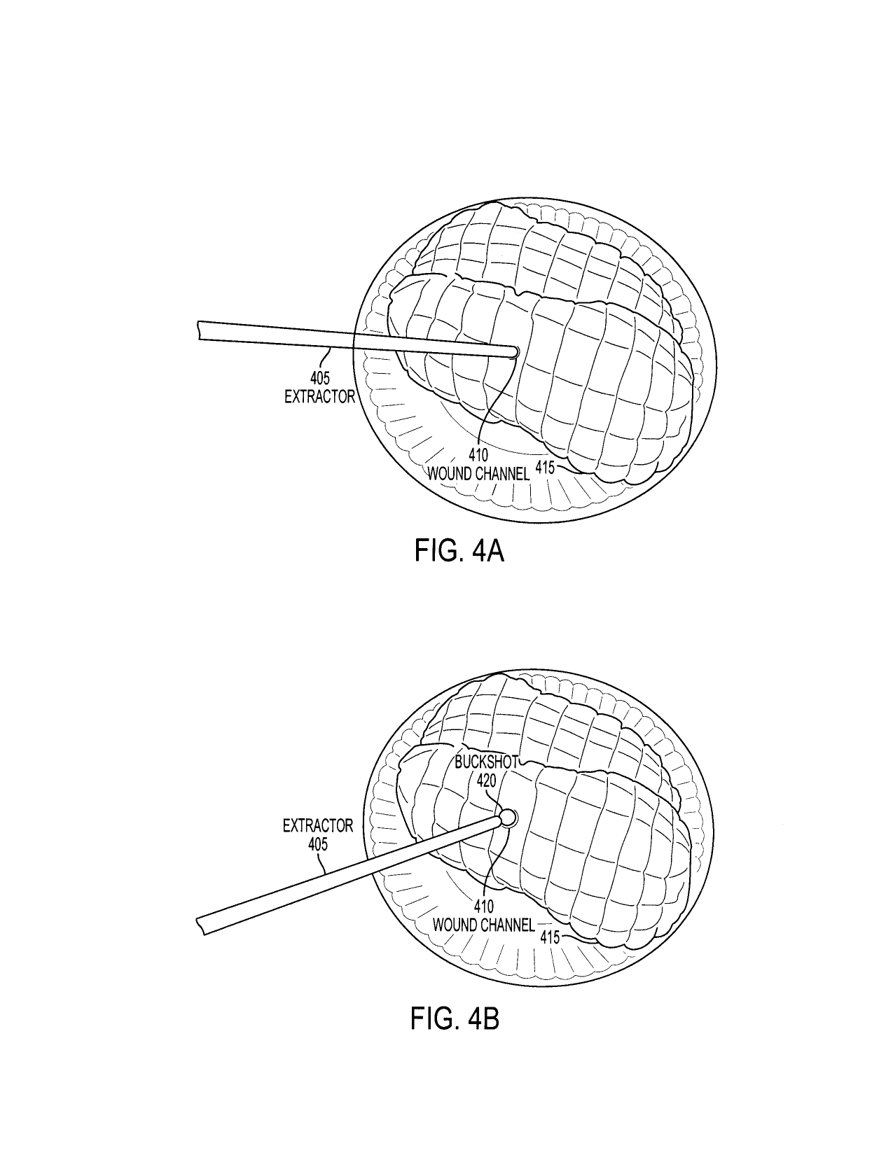

FIGS. 4A and 4B depict illustrative detection and extraction operations using an illustrative system incorporating a magnet according to an embodiment. As shown in FIG. 4A, a probe 405 may be inserted into a wound channel 410 in a subject 415. FIG. 4B shows a representative metallic object 420 attached to the probe 405 after being removed from the wound channel 410 of the subject 415.

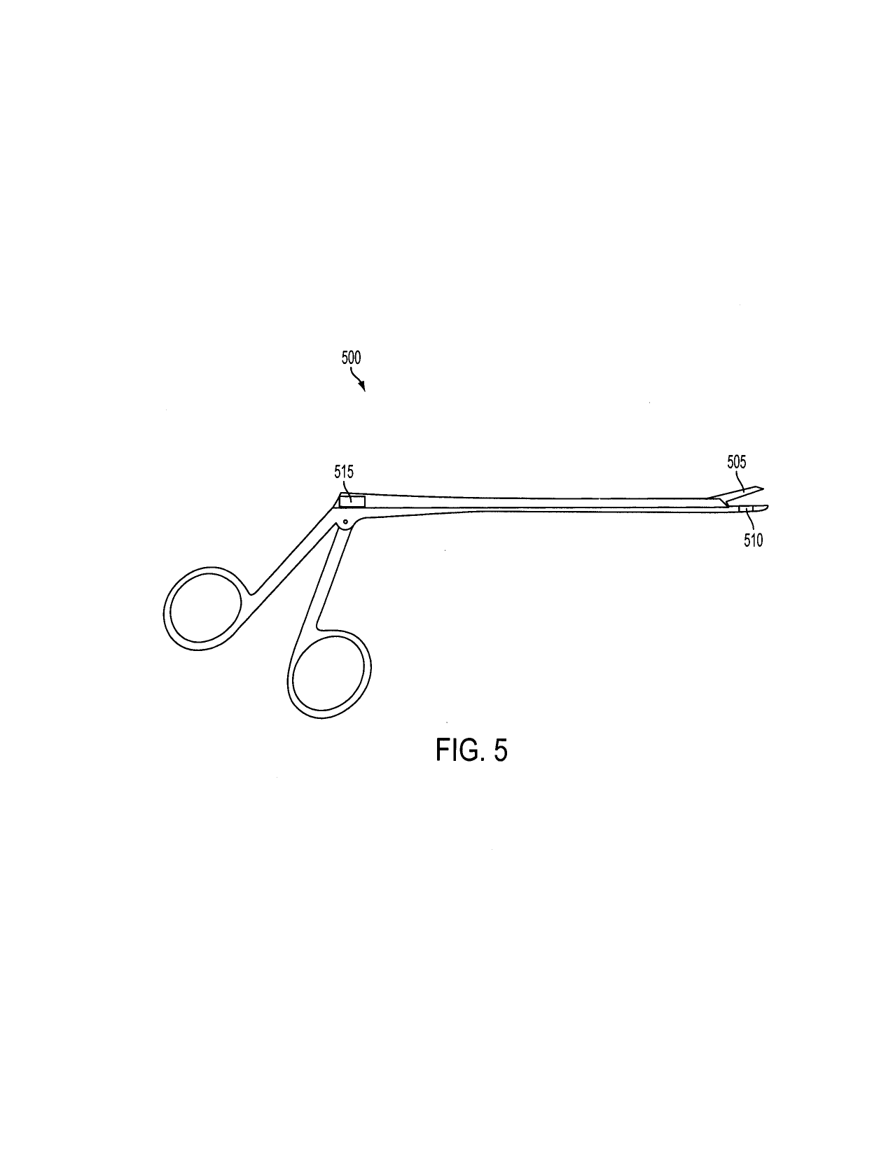

FIG. 5 depicts an alternate illustrative extraction device for detecting and removing a metallic object from a subject according to an embodiment. As shown in FIG. 5, the extraction device 500 may include a gripping portion 505, a sensor 510, and an electronics interface 515. The gripping portion 505 may be located at a distal end of the extraction device 500. The gripping portion 505 may be configured to grip a metallic object when in a closed configuration. For example, the gripping portion 505 may be placed in an open configuration in which the gripping portion may be placed around a metallic object. The gripping portion 505 may then be placed in a closed configuration in which the gripping portion closes or partially closes to affix itself to the metallic object.

The sensor 510 may be positioned at or near the distal end of the extraction device 500. In an embodiment, the sensor 510 may be positioned within the gripping portion 505 of the extraction device 500. The sensor 510 may be configured to detect a metallic object in a subject.

The electronics interface 515 may be configured to permit connection to an external control device. For example, the electronics interface 515 may include the metal detector shown in FIG. 2 and described above. In an embodiment, the electronics interface 515 may be configured to permit a physical connection the external control device. For example, a cord may be inserted into the electronics interface 515 to form a physical connection to the external control device. In an alternate embodiment, the electronics interface 515 may be configured to permit a wireless connection to be established with the external control device. A wireless connection may be formed using any commonly used circuitry and protocols including, without limitation, Bluetooth®. In an embodiment, a wireless connection could be advantageously used to connect to an external control device containing human interface elements, such as without limitation an on-off button, sensitivity control, LED and/or buzzer to signal foreign body detection. If a wireless connection were not used, all circuit components may be part of, or attached to, the foreign body extractor. In an embodiment, the electronics interface 515 may be positioned at or near a proximal end of the extraction device 500. Such an embodiment is of particular use if a physical connection is established with the external control device.

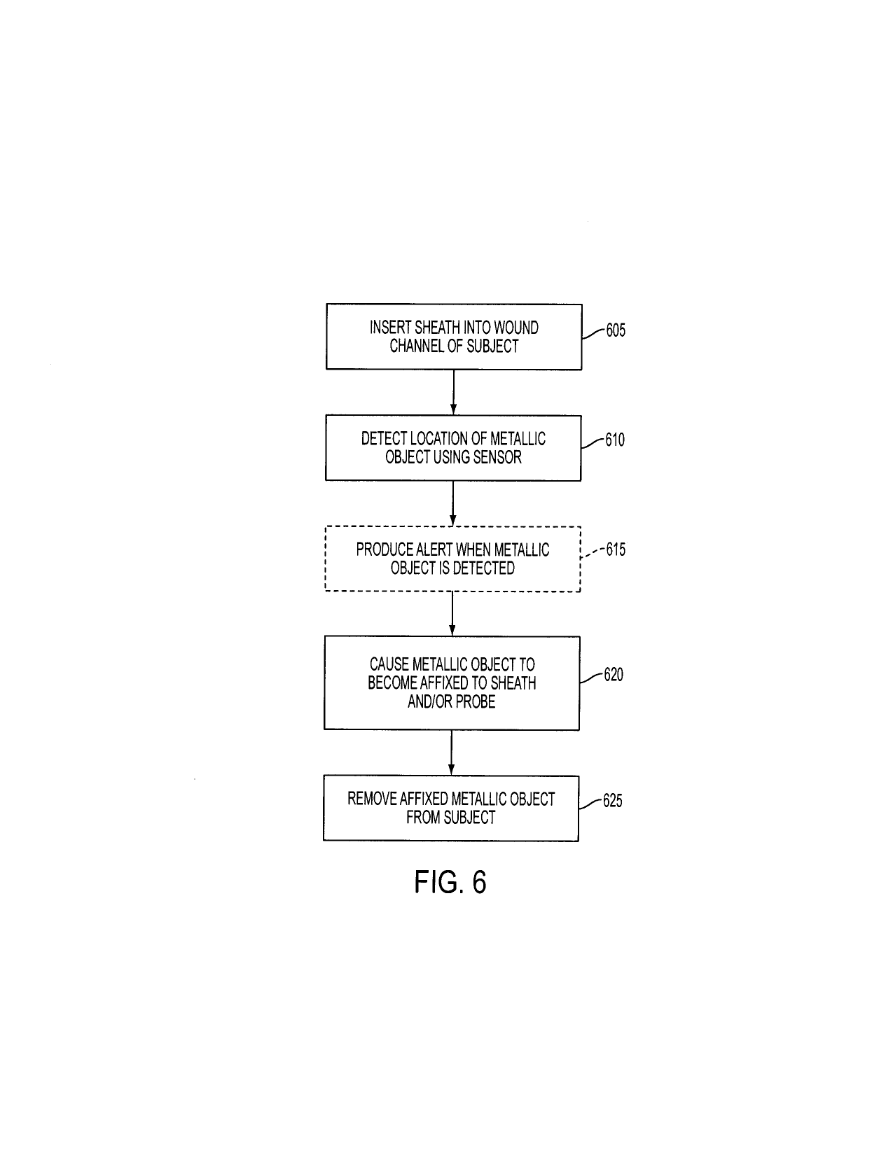

FIG. 6 depicts a flow diagram of an illustrative method of detecting and removing a metallic object according to an embodiment. As shown in FIG. 6, a sheath may be inserted 605 into a channel of a subject, such as a wound channel. The sheath may include a probe that is movably positioned within the sheath. The probe may include a sensor positioned at or near a distal end of the probe.

A location of a metallic object may be detected 610 in the subject using the sensor. In an embodiment, the probe may be positioned in an extended position during the detection 610 operation. In an embodiment, an alert may be produced 615 when the metallic object is detected. For example, an auditory alert and/or a visual alert may be produced 615. In an embodiment, detecting 610 the location of the metallic object may include producing an alert that increases in intensity as the sensor approaches the metallic object and decreases in intensity as the sensor becomes more distant from the metallic object. In an embodiment, detecting 610 the location of the metallic object may include controlling a sensitivity of the sensor. For example, the sensor may be initially configured to detect 610 metallic objects in a relatively broad volume. When the metallic object is detected 610 within this relatively broad volume, the sensitivity may be adjusted so that detection within progressively narrower volumes is required. In this manner, the location of the metallic object may be pinpointed.

The metallic object may be caused to be affixed 620 to one or more of the sheath and the probe. In an embodiment, the probe may be placed in a recessed position if the metallic object is caused to become affixed 620 to the sheath so that a better affixation may occur.

Exemplary methods of causing the metallic object to be affixed 620 to the sheath and/or the probe are described below. For example, the sheath may include a vacuum lumen through which suction is provided at the distal end of the sheath. The suction may cause the metallic object to become affixed 620 to the sheath and/or the probe for removal. The sheath may additionally or alternately include an irrigation channel through which an irrigation solution is provided. The irrigation solution may be used to dislodge the metallic object if it is embedded in tissue of the subject. Moreover, the irrigation solution may remove germs or other non-metallic debris from the wound site. If the irrigation channel and vacuum lumen are used in concert, the vacuum lumen may further suction the irrigation solution from the subject.

In an embodiment, the probe or the sheath may include a magnet positioned at the distal end of the probe or sheath. In such an embodiment, the metallic object may be caused to become affixed 620 to the magnet.

The affixed metallic object may be removed 625 from the subject. In an embodiment, the sheath may be removed from the subject to effect the removal 625 of the metallic object from the subject. In an alternate embodiment, the probe may be removed from the sheath to effect the removal 625 of the metallic object if the metallic object is affixed 620 to the probe. The latter case may be preferable if a plurality of metallic objects are believed to be located within the subject. In such a case, the sheath may remain substantially stationary during the removal 625 process, thereby causing less trauma to the wound channel of the subject.

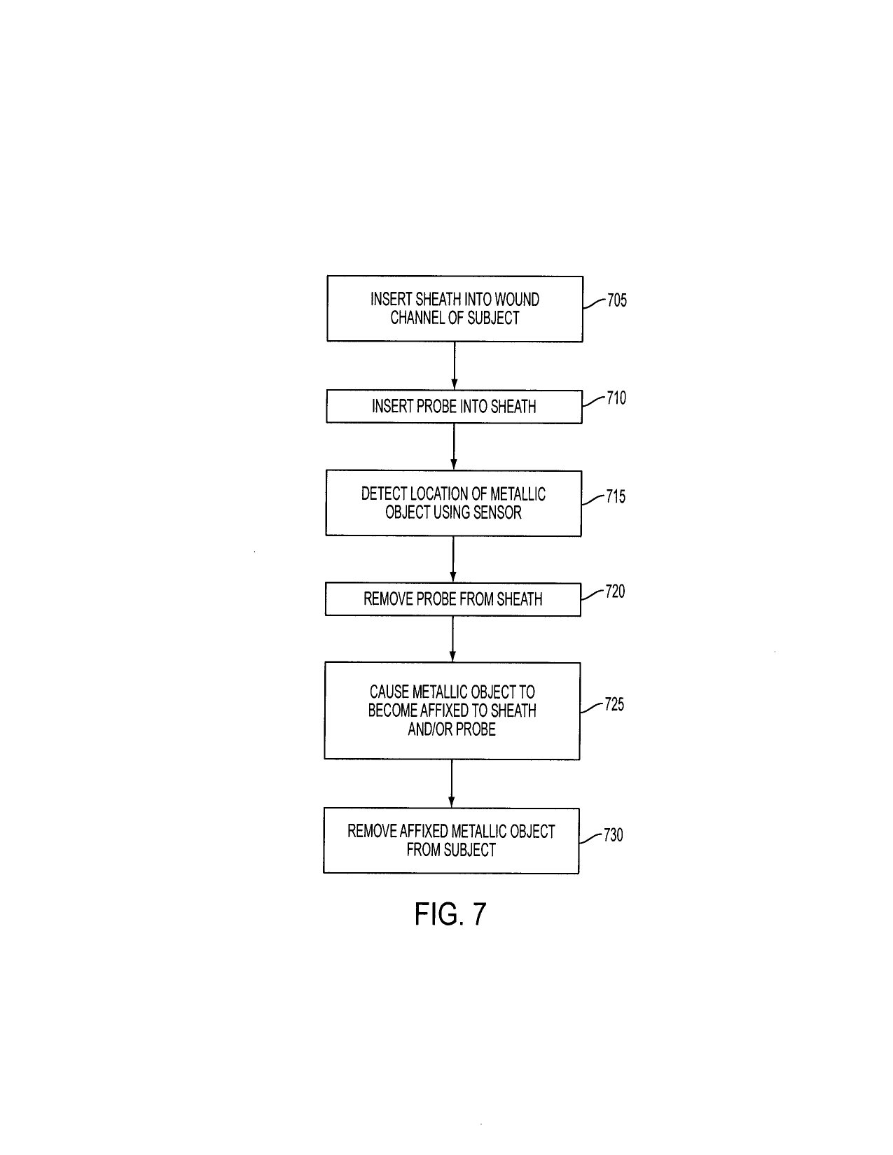

FIG. 7 depicts a flow diagram of an alternate illustrative method of detecting removing an object according to an embodiment. As shown in FIG. 7, a sheath may be inserted 705 in to a channel of a subject. A probe may be inserted 710 into the sheath. The probe may include a sensor that is positioned at or near a distal end of the probe.

A location of a metallic object in the subject may be detected 715 using the sensor. In an embodiment, the sensitivity of the sensor may be controlled as part of the process of detecting 715 the metallic object. In an embodiment, an auditory alert and/or a visual alert may be produced when the metallic object is detected. In an embodiment, an alert may be produced that increases in intensity as the sensor approaches the metallic object and decreases in intensity as the sensor becomes more distant from the metallic object.

The probe may be removed 720 from the sheath. The metallic object may be caused to become affixed 725 to the sheath. In an embodiment, the sheath may include a vacuum lumen, and the metallic object may be caused to become affixed 725 to the sheath by causing suction to be provided at the distal end of the sheath via a vacuum lumen within the sheath. In an embodiment, a device including a magnet positioned at a distal end of the device may be inserted into the sheath. In such an embodiment, the metallic object may be caused to become affixed 725 by causing the metallic object to become affixed to the magnet. The sheath and the affixed metallic object may be removed 730 from the subject.

In an embodiment, the sheath may include an irrigation channel, and an irrigation solution may be provided via the irrigation channel. In an embodiment, catheter may be inserted into the sheath that includes a vacuum lumen, and suction may be provided via the vacuum lumen. In an embodiment, a catheter may be inserted into the sheath that includes a vacuum lumen and an irrigation channel. Suction may be provided via the vacuum lumen and an irrigation solution may be provided via the irrigation channel. In an embodiment, a device including a gripping portion may be inserted into the sheath and the metallic object may be gripped using the gripping portion of the device.

In an embodiment, a kit may include a sheath configured to be inserted into a channel of a subject, a probe configured to be inserted within the sheath, and instructions for detecting and removing a metallic object from the subject using at least the probe and the sheath. The probe may include a sensor positioned at or near a distal end of the probe. In an embodiment, the kit further includes a catheter configured to be inserted within the sheath. The catheter may include a vacuum lumen and/or an irrigation channel. The kit may further include a suction device configured to be connected to the vacuum lumen. If an irrigation channel is included as part of the catheter, an irrigation source configured to be connected to the irrigation channel may also be included. In an embodiment, the kit may further include au extraction device that includes a magnet and that is configured to be inserted within the sheath. In an embodiment, the kit may further include an extraction device that includes a gripping tool and that is configured to be inserted within the sheath.

### Example 1

### Device with Magnet

A system for detecting and removing a metallic object included a sheath and a probe. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included a magnet used to affix the metallic object to the probe. Once the metallic object was affixed, the probe and/or the sheath were removed from the subject while withdrawing the affixed metallic object.

### Example 2

### Device with Gripping Tool

A system for detecting and removing a metallic object included a sheath and a probe. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included a gripping tool that was used to affix the metallic object to the probe. The gripping tool had a hinged distal end that opened and closed in response to a user's manipulation of the device. The gripping tool was opened when placed into position around the metallic object. When the metallic object was in a position to be grasped, the gripping tool was closed to affix the metallic object. The probe and/or the sheath were then removed from the subject in order to withdraw the affixed metallic object.

### Example 3

### Device Using Suction

A system for detecting and removing a metallic object included a sheath and a probe. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included a vacuum lumen and an irrigation channel. The irrigation channel was used to provide an irrigation solution at or near the location of the metallic object. The irrigation solution dislodged metallic objects that were embedded into tissue of the subject and also disinfected the wound channel. The vacuum lumen provided suction to remove the irrigation solution and to affix the metallic object to the probe and/or the sheath. Once the metallic object was affixed, the probe and/or the sheath were removed from the subject while withdrawing the attached metallic object.

### Example 4

### Sheath with Magnet

A system for detecting and removing a metallic object included a sheath, a probe and a magnet. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included depth markings allowing a user to identify the depth of the probe when the metallic object was detected. The probe was then removed from the sheath, and the magnet was inserted into the sheath. The metallic object became affixed to the magnet when the magnet was in contact with the object. The magnet and/or the sheath were then removed from the subject in order to withdraw the affixed metallic object.

### Example 5

### Sheath with Gripping Tool

A system for detecting and removing a metallic object included a sheath, a probe and an extraction device having a gripping tool. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included depth markings so that a user could identify the depth of the probe when the metallic object was detected. The probe was then removed from the sheath, and the extraction device including the gripping tool was inserted into the sheath. The gripping tool was used to affix the metallic object. The gripping tool had a hinged distal end that opened and closed in response to a user's manipulation of the device. The gripping tool was opened when placed into position around the metallic object. When the metallic object was in a position to be grasped, the gripping tool was closed to affix the metallic object. The extraction device and/or the sheath were then removed from the subject in order to withdraw the affixed metallic object.

### Example 6

### Sheath Using Suction

A system for detecting and removing a metallic object included a sheath, a probe and a catheter having two channels. The sheath was made of a material that was non-toxic and compatible with being inserted into a wound channel of a human or animal, such as a plastic. The probe included a sensor that was used to detect a metallic object. The sensitivity of the sensor was tunable to allow for an initial broader search and a progressively narrower search to identify the location of the metallic object. The probe further included depth markings so that a user could identify the depth of the probe when the metallic object was detected. The probe was removed born the sheath, and the catheter was inserted into the sheath. The two channels of the sheath supported a vacuum lumen and an irrigation channel. The irrigation channel was used to provide an irrigation solution at or near the location of the metallic object. The irrigation solution dislodged metallic objects embedded into tissue of the subject and disinfected the wound channel. The vacuum lumen provided suction to remove the irrigation solution and to affix the metallic object to the sheath. Once the metallic object was affixed, the catheter and/or the sheath were removed from the subject while withdrawing the attached metallic object.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 4, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for detecting and removing a metallic object from a subject, the system comprising:
a sheath having a distal end and configured to have the distal end inserted into a channel of the subject;
a probe configured to be movably positioned within the sheath, wherein the probe comprises a sensor configured to detect a metallic object, wherein the sensor is positioned at or near a distal end of the probe; and
a vacuum lumen positioned within the sheath, wherein the vacuum lumen is configured to provide suction at the distal end of the sheath.

2. (canceled)

3. The system of claim 1, wherein the distal end of the probe is configured to be positioned outside of the distal end of the sheath during a detection operation.

4. The system of claim 1, wherein the distal end of the probe is configured to be recessed within the sheath during a removal operation.

5. (canceled)

6. The system of claim 1, wherein the sheath comprises an elastomeric tip, wherein the elastomeric tip is positioned around at least a portion of the sensor during a detection operation and distally from the sensor during a removal operation.

7.-9. (canceled)

10. The system of claim 1, wherein the sensor is configured to cause an alert to be produced that increases in intensity as the sensor approaches the metallic object and decreases in intensity as the sensor becomes more distant from the metallic object.

11. The system of claim 1, wherein a sensitivity of the sensor is configured to be controlled by an operator.

12.-27. (canceled)

28. A system for detecting and removing a metallic object from a subject, the system comprising:
a sheath having a distal end and configured to have the distal end inserted into a channel of the subject,
wherein the sheath is further configured to removably receive a probe during a detection operation, wherein the probe comprises a sensor configured to detect a metallic object, wherein the sensor is positioned at or near a distal end of the probe.

29-32. (canceled)

33. The system of claim 28, wherein the sensor is configured to cause an alert to be produced that increases in intensity as the sensor approaches the metallic object and decreases in intensity as the sensor becomes more distant from the metallic object.

34. The system of claim 28, wherein a sensitivity of the sensor is configured to be controlled by an operator.

35.-36. (canceled)

37. The system of claim 28, wherein the probe comprises a plurality of depth markings configured to identify a depth to which the probe is inserted into the sheath.

38. The system of claim 28, wherein the sheath is further configured to receive a catheter during a removal operation, wherein the catheter comprises a vacuum lumen configured to provide a vacuum.

39. The system of claim 38, wherein the catheter further comprises an irrigation channel, wherein the irrigation channel is configured to provide an irrigation solution.

40.-43. (canceled)

44. The system of claim 28, wherein the sheath is further configured to receive an extraction device during a removal operation.

45. (canceled)

46. The system of claim 44, wherein the extraction device comprises a gripping tool at a distal end.

47. The system of claim 28, wherein the probe comprises an extraction device having a gripping tool at the distal end.

48. The system of claim 28, further comprising:
a catheter comprising a vacuum lumen configured to provide a vacuum.

49.-51. (canceled)

52. The system of claim 28, further comprising a magnet.

53. The system of claim 28, further comprising an extraction device.

54.-55. (canceled)

56. The system of claim 28, further comprising an ultrasound transducer configured to dislodge the metallic object.

57.-61. (canceled)

62. A method of detecting and removing a metallic object from a subject, the method comprising:
inserting a sheath into a channel of the subject, wherein the sheath comprises a probe movably positioned within the sheath, wherein the probe comprises a sensor positioned at or near a distal end of the probe;
detecting, via the sensor, a location of the metallic object in the subject;
causing the metallic object to become affixed to one or more of the sheath and the probe; and
removing the affixed metallic object from the subject.

63.-65. (canceled)

66. The method of claim 62, wherein detecting the location of the metallic object comprises producing an alert that increases in intensity as the sensor approaches the metallic object and decreases in intensity as the sensor becomes more distant from the metallic object.

67. (canceled)

68. The method of claim 62, wherein the sheath comprises a vacuum lumen, and wherein causing the metallic object to become affixed to the sheath comprises providing suction at the distal end of the sheath via the vacuum lumen.

69. The method of claim 62, wherein the sheath comprises an irrigation channel, and further comprising:
providing an irrigation solution via the irrigation channel.

70. The method of claim 62, wherein the probe comprises a magnet positioned at a distal end of the probe, and wherein causing the metallic object to become affixed comprises causing the metallic object to become affixed to the magnet.

71-88. (canceled)
